(19) United States
(12) Patent Application Publication
(10) Pub. No.: US 2003/0162263 A1
(43) Pub. Date: Aug. 28, 2003

(54) Peptides derived from the superantigen (SAg) ENV protein of HERV-K18 and their use in obtaining SAG-inhibitory antibodies and in vaccination against SAG

(76) Inventor: Marc Dupuis, Geneva (CH)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/236,091

(22) Filed: Sep. 6, 2002

Related U.S. Application Data
(60) Provisional application No. 60/317,703, filed on Sep. 6, 2001.
(60) Provisional application No. 60/317,704, filed on Sep. 6, 2001.

(51) Int. Cl. C12P 21/06; C12P 19/34; C07K 14/00; C07K 7/00; A61K 39/12; C07K 2/00; C07K 4/00; C07K 5/00; C07K 16/00; C07K 17/00; A61K 38/00

(52) U.S. Cl. 435/91.1; 435/91.33; 435/69.1; 424/204.1; 424/186.1; 530/300

(57) ABSTRACT

The present invention relates to peptides derived from the superantigen (SAg) ENV protein of the human endogenous retrovirus HERV-K18, and to the use of the peptides in obtaining antibodies which inhibit the superantigen activity of HERV-K18 ENV. The invention also relates to vaccine compositions for treating and preventing disorders associated with the ENV gene product of HERV-K18, for example autoimmune diseases such as insulin-dependent diabetes mellitus (IDDM). A preferred peptide consists of a portion of an N- or C-terminal segment of the HERV-K18.1 ENV protein, as illustrated in FIG. 1A, said N-terminal segment extending from amino acids 22 to 62 of HERV-K18.1 ENV, and said C-terminal segment extending from amino acids 110 to 153 of HERV-K18.1 ENV, wherein the peptide has a length of 6 to 40 amino acids and is capable of giving rise to antibodies which inhibit superantigen activity associated with HERV-K18 envelope proteins.

### RELATED APPLICATIONS

This application claims the benefit of priority under 35 U.S.C. 119(e) to copending U.S. Provisional Application No. 60/317,703, filed on Sep. 6, 2001, and No. 60/317,704, filed on Sep. 6, 2001; the entire contents of which are incorporated herein by reference. This application is also related to U.S. application Ser. No. 09/490,700, filed Jan. 24, 2000, the entire contents of which are incorporated herein by reference.

### FIELD OF THE INVENTION

The present invention relates to peptides derived from the superantigen (SAg) ENV protein of the human endogenous retrovirus HERV-K18, and to the use of the peptides in obtaining antibodies which inhibit the superantigen activity of HERV-K18 ENV. The invention also relates to vaccine compositions for treating and preventing disorders associated with the ENV gene product of HERV-K18, for example autoimmune diseases such as insulin-dependent diabetes mellitus (IDDM).

### BACKGROUND OF THE INVENTION

The human endogenous retrovirus HERV-K18 is a member of the HERV-K family. Its defective provirus has been found to be integrated in a reverse orientation in Intron I of the CD48 gene. A number of allelic variants of HERV-K18 have been identified, including the previously named IDDMK_{1.22}2 (Conrad 1997 ; International patent application WO 99/05527). The expression product of the env gene of HERV-K18 has been shown to have superantigen (SAg) activity. This SAg activity has been linked to a number of pathological states, including autoimmune diseases such as Insulin-Dependent Diabetes Mellitus (IDDM). Indeed, previous experiments have identified the HERV-K18 env gene as a candidate associated with aberrant activation of a subset of T cells found in the pancreas of individuals that succumbed to acute insulinitis [Conrad, 1997; Conrad 1994].

### SUMMARY OF THE INVENTION

Agents which specifically inhibit the superantigen activity of HERV-K18 ENV, thereby blocking the aberrant activation of pathogenic T cells, represent a novel means for preventing and treating the different disorders associated with HERV-K18, such as autoimmune diseases.

The present invention provides such inhibitory agents, namely immunogenic peptides, protective antibodies and nucleic acids encoding immunogenic peptides.

### BRIEF DESCRIPTION OF THE FIGURES

FIG. 1. 1A: Amino acid sequence of HERV-K18.1 ENV. The putative signal peptide is boxed. Preferred N- and C-terminal regions of the mature protein for generation of immunogenic peptides in accordance with the invention are underlined. Amino acid 97 shown in bold type varies according to different HERV-K18 alleles. FIGS. 1B to 1E show preferred immunogenic peptides of the invention. Numbering of amino acids includes the signal peptide (amino acids 1-21). The peptides are shown in the N- to C-terminus direction.

FIG. 2A. Inhibition of HERV-K18.1 SAG activity by rabbit polyclonal serum against C-terminal of HERV-K18 (AA 116-131). Cells are as follows:

BH B598 LCL: An EBV-transformed human B cell line;

BL41 B958: An EBV-infected Burkitt lymphoma (BL41: same Burkitt lymphoma line without infection by EBV strain B958)

A20 pCDL550: A mouse B cell line transfected with IDDMK nucleotide residues 1-550, with STOP at codon 153 (=K18.1);

A20 pCDL: same line as A20 pCDL, transfected with vector alone;

CD3 crosslink: an artificial way to induce activation of the T cell hybridomas that are specific to IDDMK or others, to show that they are indeed capable of responding to a stimulus, in this case crosslinked (immobilized) antibody directed against the CD3 subunit of the T cell receptor.

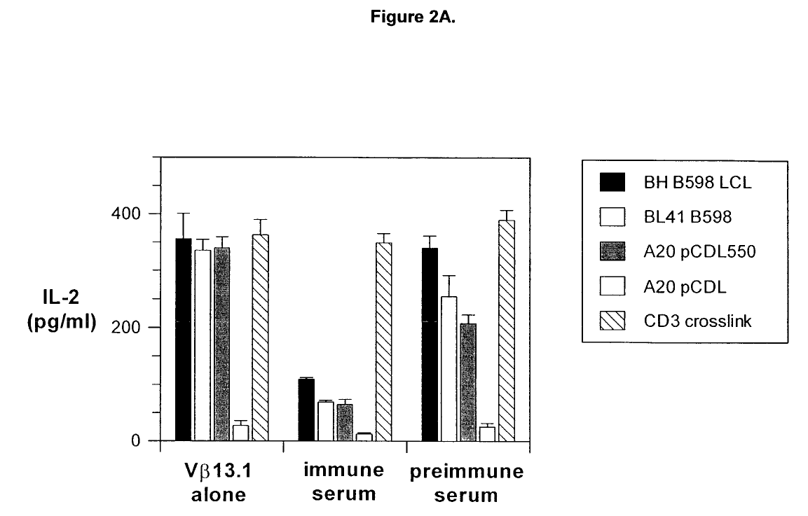

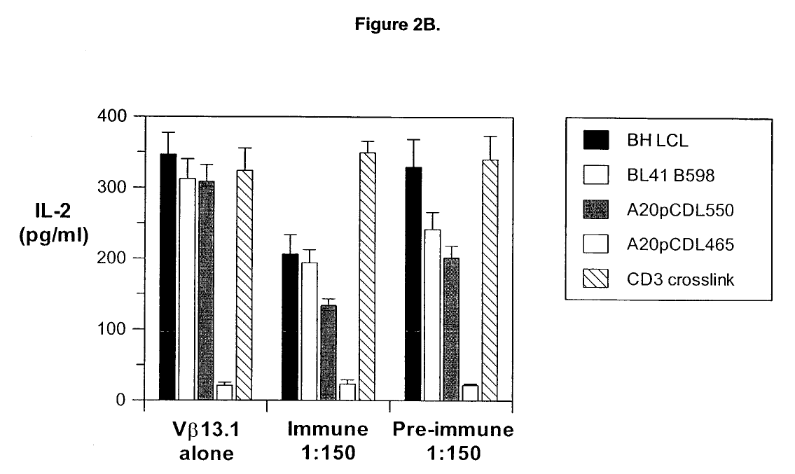

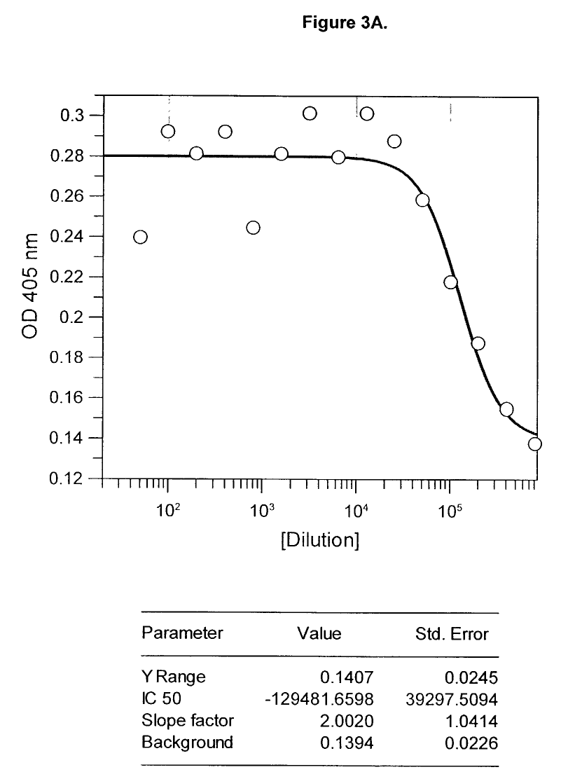

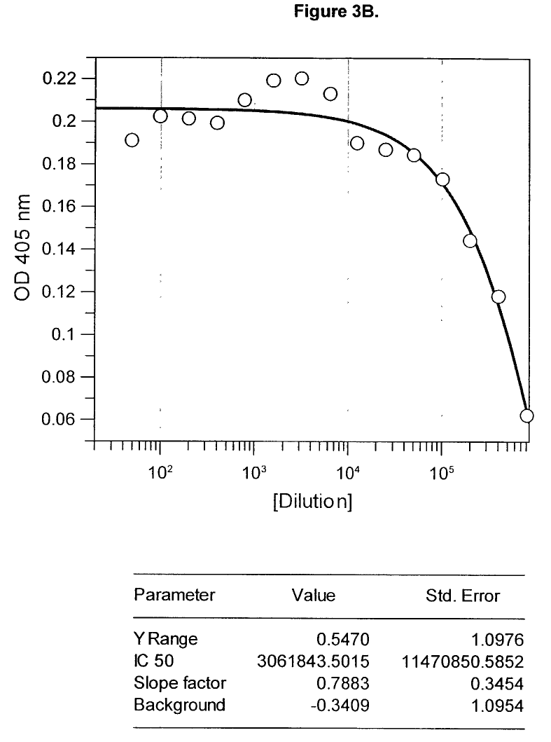

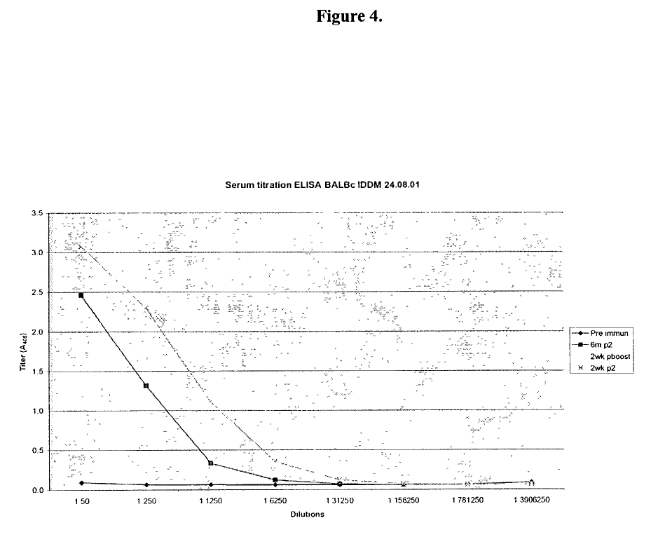

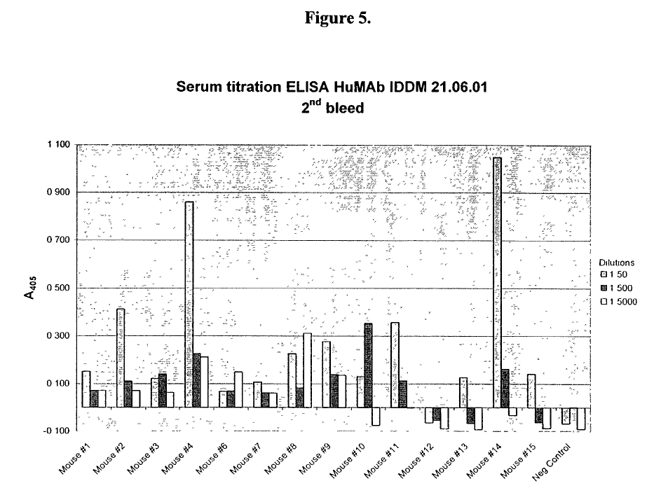

FIG. 2B. Inhibition of HERV-K18.1 SAG activity by rabbit polyclonal serum against N-terminal HERV-K18 (AA 22-32). Cells are as follows:

BH LCL: An EBV-transformed human B cell line (same as BH B598 LCL)

BL41 B958: An EBV-infected Burkitt lymphoma (BL41: same Burkitt lymphoma line without infection by EBV strain B958)

A20 pCDL550: A mouse B cell line transfected with IDDMK nucleotide residues 1-550, with STOP at codon 153 (=K18.1);

A20 pCDL 465: same line as A20 pCDL, transfected with vector alone (same as A20 pCDL);

CD3 crosslink: an artificial way to induce activation of the T cell hybridomas that are specific to IDDMK or others, to show that they are indeed capable of responding to a stimulus, in this case crosslinked (immobilized) antibody directed against the CD3 subunit of the T cell receptor.

FIG. 3A. Serum antibody titer generated against HERV-K18 AA 116-131;

FIG. 3B. Serum antibody titer generated against HERV-K18 AA 22-32;

FIG. 4. Immunization with a mixture of HERV-K18 peptides (AA 116-131; AA 113-127; AA 116-130) induces long-lasting serum titers.

FIG. 5. Induction of human antibodies in HuMAb transgenic mice immunized with a mixture of HERV-K18 peptides (AA 116-131; AA 113-127; AA 116-130) (SEQ ID:2-4)

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

In the context of the present invention, the following terms signify:

HERV-K18: a full-length defective human endogenous retrovirus localized in Intron 1 of the CD48 gene on chromosome 1. The integration site of the HERV-K18 provirus in the large first CD48 intron has been found to be preserved in all individuals tested. The provirus is inserted in the opposite transcriptional direction to CD48.

Superantigen: a substance, normally a protein, of microbial or viral origin that binds to major histocompatibility complex (MHC) Class II molecules and stimulates T-cell, via interaction with the Vβ domain of the T-cell receptor (TCR). SAgs have the particular characteristic of being able to interact with a large proportion of the T-cell repertoire, i.e., all the members of a given Vβ subset or family, or even with more than one Vβ subset, rather than with single, molecular clones from distinct Vβ families as is the case with a conventional (MHC-restricted) antigen. The superantigen is said to have a mitogenic effect that is MHC Class II dependent but MHC-unrestricted. SAgs require cells that express MHC Class II for stimulation of T-cells to occur.

Superantigen activity or SAg activity: a capacity to stimulate T-cells in an MHC-dependent but MHC-unrestricted manner. In the context of the invention, SAg activity can be detected in a functional assay by measuring either IL-2 release by activated T-cells, or proliferation of activated T-cells. Proliferation may be measured by determination of 3H-thymidine incorporation. Assays for the assessment and measurement of SAg activity are described in international patent application WO 99/05527, the content of which is hereby incorporated by reference.

“disorders associated with the superantigen activity of HERV-K 18 ENV”: any disease or disorder caused directly or indirectly by T-cell proliferation associated with superantigen activity of HERV-K18 ENV, for example those involving aberrant Vβ7 and/or Vβ13 proliferation. Such disorders include but are not limited to autoimmune disease, such as insulin-dependent diabetes mellitus (IDDM), multiple sclerosis, rheumatoid arthritis and lupus, or infectious disease or the result of an infection that may trigger the HERV-K18 SAg expression, including viral infection.

cells which functionally express SAg: cells which express SAg in a manner suitable for giving rise to MHC-dependent, MHC-unrestricted T-cell stimulation in vitro or in vivo. This requires that the cell be MHC II⁺ or that it has been made MHC II⁺ by induction by agents such as IFN-γ.

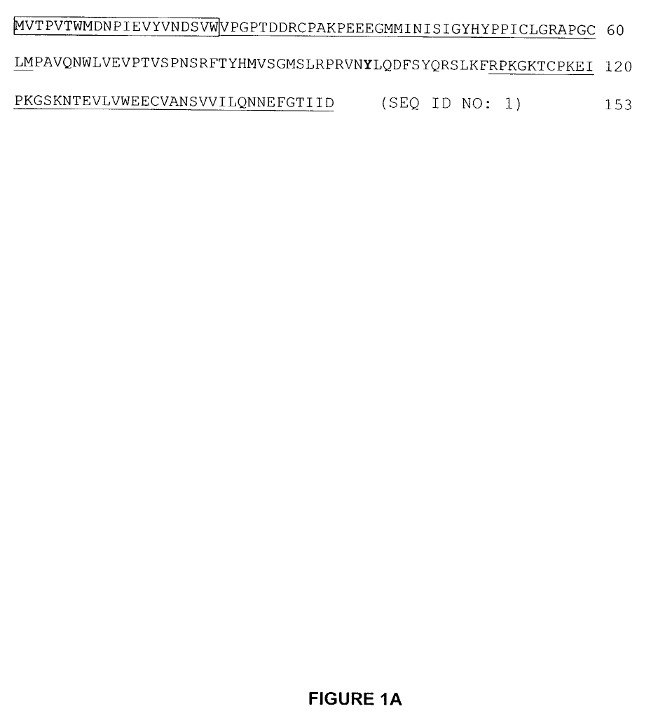

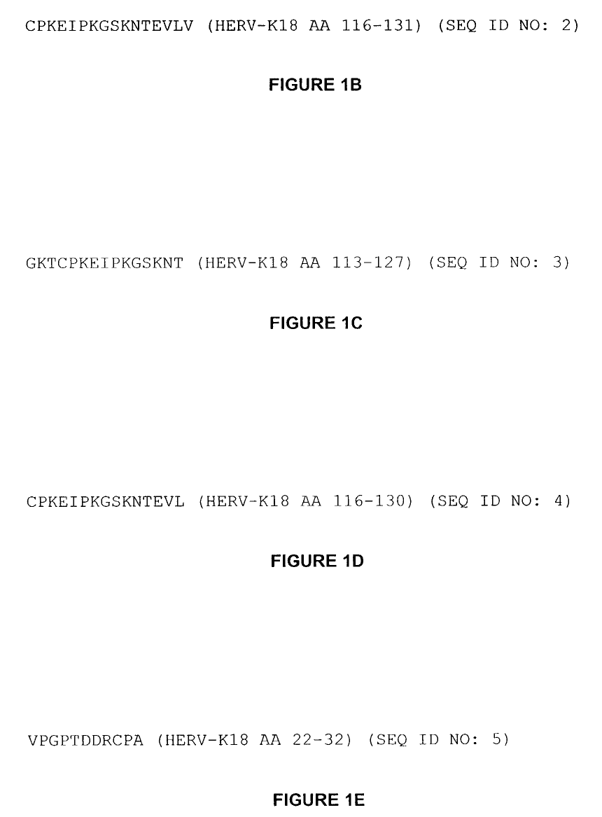

In a first embodiment, the invention provides immunogenic peptide sequences which generate protective antibodies capable of inhibiting the superantigen activity of HERVK-18 ENV. The peptides of the invention consist of fragments of the amino- and carboxy-extremities of the HERV-K18 ENV protein shown in FIG. 1A (SEQ ID NO: 1).

More specifically, the invention relates to a peptide consisting of all or part of an N- or C-terminal segment of the mature protein illustrated in FIG. 1A.

According to the invention, the C-terminal segment used as source of immunogenic peptides is the stretch of consecutive amino acids extending from amino acids 110 to 153 of the protein illustrated in FIG. 1A. Within this C-terminal region, preferred segments are those extending from amino acids 110 to 145, preferably amino acids 112 to 140, or even more preferably amino acids 112 to 135.

The N-terminal segment used as source of immunogenic peptides, is any stretch of consecutive amino acids within the region extending from amino acids 22 to 62 as illustrated in FIG. 1A. The term “N-terminal” in the context of the invention, generally speaking, signifies the N-terminus of the mature protein illustrated in FIG. 1A, i.e., without signal peptide (amino acids 1 to 21). Within this N-terminal region, preferred segments are those extending from amino acids 22 to 50, for example amino acids 22 to 42, particularly 22 to 32.

The peptide of the invention is any peptide having a length of 6 to 40 amino acids within the N- or C-terminal segments defined above. Preferably the peptide has a length of 8 to 25 amino acids and most preferably 10 to 20 amino acids, for example 10, 15, 16, 17 or 18 amino acids.

Most preferred peptides are those having a length of 10 to 18 amino acids and a sequence selected from the N-terminal region extending from amino acids 22 to 40, or from the C-terminal region extending from amino acids 112 to 135, particularly 113 to 132.

Examples of preferred peptides of the invention are those which comprise or consist of:

amino acids 22 to 32 of the protein illustrated in FIG. 1A: VPGPTDDRCPA (HERV-K18 AA 22-32)

amino acids 116 to 131 of the protein illustrated in FIG. 1A: CPKEIPKGSKNTEVLV (HERV-K18 AA 116-131)

amino acids 116 to 130 of the protein illustrated in FIG. 1A: CPKEIPKGSKNTEVL (HERV-K18 AA 116-130)

amino acids 113 to 127 of the protein illustrated in FIG. 1A: GKTCPKEIPKGSKNT (HERV-K18 AA 113-127)

Particularly efficient peptides derived from the C-terminal region of the HERV-K18 ENV protein comprise the sequence CPKEIPKGSKNT. Preferred peptides of this type have a length of 15 to 40 amino acids, preferably 15 to 18 amino acids, and comprise at least 3 further amino acids on the N-terminal extremity of the CPKEIPKGSKNT sequence (for example HERV-K18 AA 113-127), or at least 4 further amino acids on the C-terminal extremity of the CPKEIPKGSKNT sequence (for example HERV-K18 AA 116-131).

Preferred proteins are illustrated in FIGS. 1B (SEQ ID NO: 2), 1C (SEQ ID NO: 3), 1D (SEQ ID NO: 4) and 1E (SEQ ID NO: 5) respectively. Other preferred proteins are longer peptides which contain the above peptides in their sequence and which have a maximum length of around 30 amino acids, preferably 20 amino acids.

The peptides of the invention elicit antibodies which inhibit superantigen activity associated with HERV-K18 envelope proteins. In the context of the invention, the term “HERV-K18 ENV proteins” means any HERV-K18 envelope allele, including HERV-K18.1 (WO 99/05527), HERV-K18.2 (Tönjes et al., 1999) and HERV-K18.3 (Conrad et al., 2001), and any derivatives or variants of the naturally occurring HERV-K18 ENV proteins which substantially conserve the properties of the natural proteins. The sequence of the HERV-K18.1 ENV allele is shown in FIG. 1A, including the 21 amino-acid signal peptide (boxed). The K18.1 ENV allele is a 153 amino-acid truncation of the full-length HERV-K18.2 and HERV-K18.3 alleles, which have 560 amino acids. The regions of the HERV-K18.1 allele which are designated “N- and C-terminal segments” in the present invention are common to all alleles.

In the context of the invention, “superantigen activity associated with HERV-K18 envelope proteins” means any superantigen activity exhibited in vitro or in vivo by any HERV-K18 envelope allele, including HERV-K18.1, HERV-K18.2 and HERV-K18.3 and fragments, derivatives and variants of the HERV-K18 envelope proteins. Superantigen activity is the capacity to stimulate T-cells in an MHC-Class II dependent but MHC unrestricted manner i.e., a superantigen induces proliferation of all the members of a given Vβ subset of the T-cell repertoire, or even several Vβ subsets. The HERV-K18 ENV proteins generally exhibit Vβ7 and Vβ13 SAg activity. Assays for the assessment and measurement of such activity are described in international patent application WO 99/05527, the content of which is hereby incorporated by reference.

According to the invention, the “capacity to inhibit SAg activity associated with HERV-K18 ENV proteins” signifies that SAg activity of the ENV proteins as defined above is reduced or eliminated or that onset of detrimental T-cell proliferation is retarded. In vitro, the inhibitory capacity can be tested or demonstrated using the transfectant assays described in the examples below. For example, the inhibitory activity of the antibodies generated by the immunogenic peptides of the invention can be tested in vitro by introducing the antibodies under test into an assay system comprising:

i) MHC Class II⁺ cells functionally expressing HERV-K ENV protein having SAg activity and

ii) cells bearing Vβ-T cell receptor chains of the family or families specifically stimulated by the HERV-K18 SAg expressed by the MHC Class II⁺ cells (particularly Vβ-7 and/or Vβ-13), and determining the capacity of the antibodies under test to diminish or block Vβ-specific stimulation by the HERV-K18 SAg.

The cells bearing the V-β receptor chains may be either an unselected population of T-cells or T-cell hybridoma. The MHC Class II+ cells may be B-lymphocytes, monocytes, macrophages or dendritic cells which have the capacity to bind the superantigen and enable it to elicit its superantigen activity. MHC class II content of the biological sample may be boosted by addition of agents such as IFN-gamma. Read-out is typically IL-2 release or alternatively ³H-thymidine incorporation.

Generally speaking, in such in vitro assays, the agents of the invention exhibit a capacity to inhibit SAg activity by at least 40%, preferably at least 50%, and most preferably at least 70%, for example at least 80% or 85%. Such inhibition can be achieved at antibody dilutions of 1:100, 1:150 and 1:200. Particularly preferred peptides such as HERV-K18 AA 116-131, and HERV-K18 AA 113-127, or mixtures thereof additionally including HERV-K18 AA 116-130, give rise to sera exhibiting at least approximately 55% inhibition, and more preferably at least approximately 75% or at least 80% inhibition at dilutions of 1:100 or 1:150. The inhibitory agents of the invention inhibit Vβ7 and/or Vβ13 activity, preferably both Vβ7 and Vβ13. The inhibitory effect of the antibodies is elicited without the need to immunopurify the antibodies.

The inhibitory capacity of the antibodies raised to the peptides of the invention can also be tested in vivo. Animals are immunized with the immunogenic compositions of the invention then tested for the effect of SAg in vivo by a standard assay, namely the injection of MHC class II positive SAg transfectants (such as the transfectants described in the examples below), known to induce a Vβ-specific T cell activation in vivo. Successful immunization against a HERV-K18 ENV SAg protein results in a reduction or in a block of the in vivo SAg-induced T cell activation and proliferation in effectively immunized individuals. This procedure is referred to as anti- HERV-K18 SAg vaccination. Immunization against HERV-K18 SAg can be performed in a therapeutic setting in humans, for example for diabetes, preferably initially in the case of early diagnosed IDDM patients. Efficacy of this novel vaccination procedure is monitored by clinical outcome and by reduction of the expected requirements for insulin therapy. In the case of other disorders associated with HERV-K18 ENV, the clinical outcome is monitored accordingly.

The invention further relates to immunogenic compositions comprising a peptide of the invention, or a mixture of such peptides. Mixtures of peptides are particularly preferred. The mixtures may contain both N- and C-terminal peptides, or may contain only N- or only C-terminal peptides. According to a preferred variant, the composition comprises at least two, for example three or four immunogenic peptides from the C-terminal segment of the ENV protein, particularly from the region extending from amino acids 110 to 145.

The invention also relates to fusion proteins comprising an immunogenic peptide as described above, fused to a heterologous peptide, wherein “heterologous” means a protein different from the HERV-K18 ENV protein. Such heterologous protein may be used for a variety of reasons, for example to enhance the immune response, or to provide a second function to the protein such as a targeting function, stabilization etc.

According to a second embodiment of the invention, the inhibitory agent comprises antibodies inhibiting SAg activity associated with HERV-K18 envelope proteins. Such antibodies are capable of specifically recognizing an immunogenic peptide as defined above, and can be produced using such peptides as immunogen in a suitable animal. The antibodies are capable of inhibiting Vβ7 and/or Vβ13 SAg activity, and when used in vivo generally inhibit SAg activity from both alleles of the HERV-K 18 env gene.

The antibodies of the invention may be polyclonal or monoclonal. They may be human or non-human. Monoclonal antibodies may be fully or partially humanized.

To elicit the protective effect, the antibodies of the invention may be raised in vivo against the immunogenic peptides. Alternatively the protective antibodies may be used in a method of passive transfer to an individual susceptible to disease triggered by HERV-K18env.

The invention also relates to nucleic acid molecules encoding an immunogenic peptide as defined above. Such nucleic acids may be used to generate recombinant immunogenic peptides in vitro, or alternatively, may be used in vivo as part of vaccinating compositions. The nucleic acid may be RNA or DNA.

Vaccine compositions comprising an immunogenic peptide of the invention, or a mixture of such peptides, in association with a pharmaceutically acceptable carrier are also within the scope of the invention.

Similarly, the invention also relates to vaccine compositions comprising nucleic acid molecules encoding an immunogenic peptide, in association with a pharmaceutically acceptable carrier.

The invention also extends to compositions containing protective antibodies as described above, or a mixture of said antibodies, in association with a pharmaceutically acceptable carrier. Such compositions can be used in passive transfer of immunity.

In accordance with the invention, the vaccine compositions or antibody compositions are administered to a subject for treating or preventing disorders associated with superantigen activity of HERV-K18 envelope proteins. Such disorders include autoimmune disease, such as insulin-dependent diabetes mellitus (IDDM), multiple sclerosis, rheumatoid arthritis and lupus. The disorder may also be an infectious disease or the result of an infection that may trigger the HERV-K18 SAg expression, including viral infection. Indeed, the vaccinating compositions of the invention may be used in the treatment or prevention of any T-cell proliferation-related disorders, particularly those involving aberrant Vβ7 and/or Vβ13 proliferation. Prevention and treatment of such a disorder includes prevention or delay of onset of disease or its clinical symptoms, and reduction or elimination of symptoms.

The vaccines or antibody compositions of the invention can be prepared as injectables, e.g. liquid solutions or suspensions. Solid forms for solution in, or suspension in, a liquid prior to injection also can be prepared. Optionally, the preparation also can be emulsified. The active antigenic ingredient or ingredients can be mixed with excipients which are pharmaceutically acceptable and compatible with the active ingredient. Examples of suitable excipients are water, saline, dextrose, glycerol, ethanol, or the like, and combinations thereof. In addition, if desired, the vaccine can contain auxiliary substances such as wetting or emulsifying agents, pH buffering agents, or adjuvants such as aluminum hydroxide or muramyl dipeptide or variations thereof. In the case of peptides, coupling to larger molecules (e.g. KLH or tetanus toxoid) sometimes enhances immunogenicity. The vaccines are conventionally administered parenterally, by injection, for example, either subcutaneously or intramuscularly. Additional formulations which are suitable for other modes of administration includes suppositories and, in some cases, oral formulations.

The invention also relates to the use of the inhibitory agent, i.e., the peptides, vaccine compositions or antibody compositions of the invention, in the manufacture of a medicament for inhibition of superantigen activity, or for the treatment or prevention of disorders associated with superantigen activity of HERV-K18 envelope proteins.

### EXAMPLES

### Example 1

Identification of Immunogenic Peptide Sequences of HERV-K18 env

Amino acid sequences for generating immunogenic peptides include the entire HERV-K18 ENV protein (SEQ ID NO: 1; FIG. 1A: K18.1), and fragments thereof, including AA 116-131, AA 113-127, AA116-130, AA 22-32 (FIGS. 1B (SEQ ID NO:2), 1C (SEQ ID NO:3), 1D (SEQ ID NO:4), 1E (SEQ ID NO:5)). Immunogenic peptides were identified using the hydrophilicity index of Kyte and Doolittle. Amongst the immunogenic peptides, AA 116-131, AA 113-127, AA116-130, AA 22-32 were found to be capable of inhibiting SAg activity of HERV-K18 ENV.

### Example 2

Inhibition of HERV-K18 Superantigen Activity by Serum Directed Against Immunogenic Peptides of HERV-K18

Polyclonal antibodies blocking the superantigen activity of HERV-K18 env were obtained by injecting immunogenic peptides coupled to an adequate carrier and mixed with adjuvant into rabbit, mice, or transgenic mice expressing human immunoglobulins.

Polyclonal serum antibodies were tested for their ability to block superantigen activity of the HERV-K18 env protein (HERV-K18.1) in an in vitro assay (FIG. 2). BH LCL, BL41B958, and A20 cells expressing the env protein of HERV-K18 were used to stimulate using T cells expressing the human Vβ13.1 T cell receptor. A rabbit polyclonal serum directed against HERV-K18 amino acid 116-131 was able to specifically inhibit superantigen activity by 80% at a dilution at 1:150 (as shown below in Table I and graphically in FIG. 2A).

Table I
Inhibition of Vβ13.1 IL-2 release
with serum against aa 116-131 of SEQ ID NO: 1 (%)[Dilution 1:150]

| | | | | | CD3 |
|---|---|---|---|---|---|
| | BH B598 | | | | cross- |
| | LCL | BL41B958 | A20 pCDL550 | A20 pCDL | link |
| immune | 69 | 79 | 81 | 55 | 4 |
| Pre- | 4 | 24 | 39 | 6 | −8 |
| immune | | | | | |

The polyclonal antibodies were able to inhibit superantigen activity at dilutions of up to 1:200 without the requirement of immuno-purifying the serum.

A rabbit polyclonal serum directed against HERV-K18 amino acid 22-32 was also able to specifically inhibit superantigen activity although to a lesser extent (as shown below in Table II and graphically in FIG. 2B).

Table II
Inhibition of Vβ13.1 IL-2 release
with serum against aa 22-32 of SEQ ID NO: 1 (%) [Dilution 1:150]

| | | | | | CD3 |
|---|---|---|---|---|---|
| | | | | A20 pCDL | cross- |
| | BH LCL | BL41B958 | A20 pCDL550 | L456 | link |
| immune | 41 | 38 | 67 | −11 | −7 |
| Pre- | 5 | 23 | 35 | −4 | −5 |
| immune | | | | | |

A similar inhibition of HERV-K18.1 SAG activity could be obtained when a mixture of HERV-K18 peptides (AA 116-131, AA 113-127, AA 116-130) was used as antigen for generating polyclonal serum in mice.

Similar results were obtained when inhibition of Vβ7 superantigen activity was measured using the same peptides and mixture (data not shown).

### Example 3

Immunization (vaccination) with HERV-K18 Peptides Induces Strong and Long-lasting Antibody Titers

Immunization with HERV-K18 peptides induces high serum antibody titers in mice (FIG. 3). Mice were immunized twice with 2 consecutive doses of HERV-K18 peptides coupled to a KLH carrier and mixed to Freund's adjuvant. Specific serum antibody titers for were measured 2 weeks after the second injection. Serum antibody titers specific for HERV K18 peptides corresponding to AA 116-131 and AA 22-32 were both above the 100,000-fold dilution (FIG. 3A and 3B).

Immunization with a mixture of HERV-K18 peptides induced a long-lasting immune response in mice (FIG. 4). Mice were immunized with a mixture of peptides corresponding to HERV-K18 AA 116-131, AA 113-127 and AA116-130. Serum antibody titers were measured 2 weeks after the second injection (2 wk p2), 6 months after the second injection (6 mo p2), and 2 weeks after a boost that was scheduled 6 months after the second injection (2 wk per boost; see FIG. 4). Specific antibody titers were detected 6 months after the second injection. The antibody titer was increased when a subsequent injection (Boost) was administered. This experiment shows that the peptide mixture can be used to generate long-lasting serum antibodies, an important feature of efficacy and protection in vaccination.

Serum antibody titers could also be measured in transgenic mice that express the human repertoire of antibodies (FIG. 5). Transgenic mice were immunized with a mixture of peptides corresponding to HERV-K18 AA 116-131, AA 113-127, and AA 116-130. Positive serum antibody titers were detected 2 weeks after the third injection. This experiment suggests that the peptide mixture could be used to raise HERV-K18 specific antibody titers in human.

References

1. Conrad, B., E. Weidmann, G. Trucco, W. A. Rudert, R. Behboo, C. Ricordi, H. Rodriquez-Rilo, D. Finegold, and M. Trucco. 1994. Evidence for superantigen involvement in insulin-dependent diabetes mellitus etiology. Nature 371:351.

2. Conrad, B., R. N. Weissmahr, J. Boni, R. Arcari, J. Schupbach, and B. Mach. 1997. A human endogenous retroviral superantigen as candidate autoimmune gene in type I diabetes. Cell 90:303.

3. Tönjes R, Czauderna F, Kurth R, Genome-wide screening, Cloning, Chromosomal Assignment and Expression of Full-Length Human Endogenous Retrovirus Type K. J. Virology, November 1999, 9187-9195.

4. Conrad B, et al., Immunity, 15, 591-601, October 2001.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications considered to be within the scope of the following claims. The claims presented are representative of the inventions disclosed herein. Other, unclaimed inventions are also contemplated. Applicants reserve the right to pursue such inventions in later claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A peptide comprising a portion of an N- or C-terminal segment of SEQ ID NO:1,
said N-terminal segment extending from amino acids 22 to 62 of SEQ ID NO: 1;
said C-terminal segment extending from amino acids 110 to 153 of SEQ ID NO: 1, and
wherein said peptide has a length of 6 to 40 amino acids and is capable of giving rise to antibodies which inhibit superantigen activity associated with HERV-K18 envelope proteins.

2. The peptide of claim 1, having from 8 to 25 amino acids.

3. The peptide of claim 1, having from 10 to 20 amino acids.

4. The peptide of claim 1, wherein the N-terminal segment thereof extends from amino acids 22 to 50 of SEQ ID NO: 1.

5. The peptide of claim 1, wherein the N-terminal segment thereof extends from amino acids 22 to 42 of SEQ ID NO: 1.

6. The peptide of claim 1, wherein the C-terminal segment thereof extends from amino acids 110 to 145 of SEQ ID NO: 1.

7. The peptide of claim 1, wherein the C-terminal segment thereof extends from amino acids 112 to 140 of SEQ ID NO: 1.

8. The peptide of claim 1, wherein the C-terminal segment thereof extends from amino acids 112 to 135 of SEQ ID NO: 1.

9. The peptide of claim 1, comprising amino acids 22 to 32 of SEQ ID NO: 1.

10. The peptide of claim 1, comprising amino acids 116 to 131 of SEQ ID NO: 1.

11. The peptide of claim 1, comprising amino acids 116 to 130 of SEQ ID NO: 1.

12. The peptide of claim 1, comprising amino acids 113 to 127 of SEQ ID NO: 1.

13. The peptide of claim 1, which is capable of giving rise to antibodies which inhibit Vβ7 and/or Vβ13 SAg activity.

14. Antibodies specifically recognizing the peptide of claim 1, wherein said antibodies are capable of inhibiting SAg activity associated with HERV-K18 envelope proteins.

15. The antibodies of claim 14, which are capable of blocking Vβ7 and/or Vβ13 SAg activity.

16. The antibodies of claim 14, which are capable of blocking SAg activity from both alleles of the HERV-K 18 ENV gene in vivo.

17. The antibodies of claim 14, which are polyclonal.

18. The antibodies of claim 14, which are monoclonal.

19. The antibodies of claim 14, which are human or humanized.

20. A nucleic acid encoding the peptide of claim 1.

21. An immunogenic composition comprising the peptide of claim 1, or a mixture of peptides of claim 1.

22. A vaccine composition comprising the peptide of claim 1, or a mixture of peptides of claim 1, and a pharmaceutically acceptable carrier.

23. A vaccine composition comprising the nucleic acid of claim 20, and a pharmaceutically acceptable carrier.

24. A pharmaceutical composition comprising the antibodies of claim 14, or a mixture of said antibodies, and a pharmaceutically acceptable carrier.

25. A method for inhibiting superantigen activity associated with HERV-K18 envelope proteins in a subject, comprising administering the vaccine composition of claim 22 or the pharmaceutical composition of claim 24 to a subject.

26. A method for inhibiting superantigen activity associated with HERV-K18 envelope proteins in a subject, comprising administering the vaccine composition of claim 23 or the pharmaceutical composition of claim 24 to a subject.

27. A method for treating or preventing disorders associated with superantigen activity of HERV-K18 envelope proteins in a subject in need of such treatment, said method comprising the administration of the vaccine composition of claim 22, or the pharmaceutical composition of claim 24, to a subject.

28. A method for treating or preventing disorders associated with superantigen activity of HERV-K18 envelope proteins in a subject in need of such treatment, said method comprising the administration of the vaccine composition of claim 23, or a pharmaceutical composition of claim 24, to a subject.

29. The method of claim 27, wherein said disorder is an autoimmune disease.

30. The method of claim 29, wherein said disorder is insulin-dependent diabetes mellitus.

31. The method of claim 27, wherein said disorder is the result of a bacterial or viral infection.

32. A method for treating T-cell proliferation-related disorders in a subject, comprising the administration of the composition of claim 22 to a subject.

33. A method for treating T-cell proliferation-related disorders in a subject, comprising the administration of the composition of claim 23 to a subject.

34. A method for treating T-cell proliferation-related disorders in a subject, comprising the administration of the composition of claim 24 to a subject.


SEQUENCE LISTING (5 sequences)

SEQ ID NO: 1 - protein, 153 aa, Human endogenous retrovirus
MVTPVTWMDNPIEVYVNDSVWVPGPTDDRCPAKPEEEGMMINISIGYHYPPICLGRAPGCLMPAVQNWLVEVPTVSPNSRFTYHMVSGMSLRPRVNYLQDFSYQRSLKFRPKGKTCPKEIPKGSKNTEVLVWEECVANSVVILQNNEFGTIID

SEQ ID NO: 2 - protein, 16 aa, Human endogenous retrovirus
CPKEIPKGSKNTEVLV

SEQ ID NO: 3 - protein, 15 aa, Human endogenous retrovirus
GKTCPKEIPKGSKNT

SEQ ID NO: 4 - protein, 15 aa, Human endogenous retrovirus
CPKEIPKGSKNTEVL

SEQ ID NO: 5 - protein, 11 aa, Human endogenous retrovirus
VPGPTDDRCPA